(19) United States
(12) Patent Application Publication
(10) Pub. No.: US 2012/0053597 A1
(43) Pub. Date: Mar. 1, 2012

(54) MOBILE ROBOTIC SURGICAL SYSTEM

(75) Inventors: Mehran Anvari, Hamilton (CA); David Williams, Oakville (CA); John Lymer, Toronto (CA)

(73) Assignee: MCMASTER UNIVERSITY, Hamilton, ON (CA)

(21) Appl. No.: 13/255,886

(22) PCT Filed: Mar. 10, 2010
(86) PCT No.: PCT/CA2010/000314

§ 371 Date: Nov. 17, 2011

Related U.S. Application Data
(60) Provisional application No. 61/158,852, filed on Mar. 10, 2009.

(51) Int. Cl. A61B 19/00

(52) U.S. Cl. 606/130

(57) ABSTRACT

A mobile robotic surgical system which includes a mobile surgical robot for use in a mobile or confined environment and a control station in communication with the mobile surgical robot over a network. The mobile surgical robot includes a controller for controlling operation of the mobile surgical robot, a communications subsystem for communicating with the control station over the network, robotic surgical instruments controllable by the control station over the network, a detector subsystem for determining spatial information relating to a surgical environment of the surgical robot, and a motion stabilizer subsystem for facilitating operation of the robotic surgical instruments while the mobile environment is in motion. The controller is configured to operate a local control loop between at least one of the subsystems and the robotic surgical instruments.

### CROSS-REFERENCE TO RELATED APPLICATION

This application claims the benefit of and priority to U.S. Provisional Patent Application No. 61/158,852 filed Mar. 10, 2009 under the title MOBILE ROBOTIC SURGICAL SYSTEM.

The content of the above patent application is hereby expressly incorporated by reference into the detailed description hereof.

### FIELD

Some example embodiments described herein relate to surgical robotics, and in particular to robotic surgical systems in a mobile environment.

### BACKGROUND

Surgery robotics has been a developing field. In some first-generation surgical robots, the absence of force feedback has limited the surgeon's natural sense of manipulating tissues manually. The addition of haptic feedback to the hand controllers in the surgeon's console has been available to give surgeons a sense of the amount of force applied to tissue during surgical manipulation.

There are difficulties in developing technologies to support robotic surgery in a moving vehicle.

An example difficulty which could arise is network latency, wherein the robotic system may temporarily or intermittently lose communication with the base station.

Another example difficulty is that vibrations, bumps, and gyrations may occur when a vehicle is moving.

Another example is that such difficulties could arise in emergency medical applications, wherein off-site surgery away from a medical facility could be advantageous when the patient has a time-sensitive condition or trauma.

### SUMMARY

It would be advantageous to provide a portable robotic surgical system incorporating image guidance that can be used in a wide range of clinical circumstances. It would be advantageous to provide a portable robotic surgical system to be used for computer assisted surgical procedures, including semi-autonomous and autonomous capability for the surgical robotic system for use in a mobile environment.

In an example embodiment, there is generally provided a mobile surgical robot for use in a mobile or confined environment and in communication with a control station located remotely to the surgical robot. The mobile surgical robot includes a controller for controlling operation of the mobile surgical robot, one or more subsystems, and robotic surgical instruments controllable by the control station over the network. The controller is configured to operate a local control loop between at least one of the subsystems and the robotic surgical instruments.

In another example embodiment, there is provided a mobile surgical robot, including: a controller for controlling operation of the mobile surgical robot; a communications subsystem for communicating over a network with a control station located remotely to the mobile surgical robot; robotic surgical instruments controllable by the control station over the network; a detector subsystem for determining spatial information relating to a surgical environment of the mobile surgical robot; and a motion stabilizer subsystem for facilitating operation of the robotic surgical instruments while the mobile surgical robot is in motion, wherein the controller is configured to operate a local control loop between at least one of the subsystems and the robotic surgical instruments.

In another example embodiment, there is provided a method for controlling a mobile surgical robot. The method includes: controlling operation of the mobile surgical robot using a controller; communicating with a control station located remotely to the mobile surgical robot over a network using a communications subsystem; receiving commands from the control station over the network for controlling robotic surgical instruments of the mobile surgical robot; determining spatial information relating to a surgical environment of the mobile surgical robot using a detector subsystem; facilitating operation of the robotic surgical instruments while the mobile surgical robot is in motion using a motion stabilizer subsystem; and operating a local control loop between at least one of the subsystems and the robotic surgical instruments using the controller.

In another example embodiment, there is provided a mobile robotic surgical system, comprising a mobile surgical robot and a control station located remotely to the mobile surgical robot in communication with the mobile surgical robot over a network. The mobile surgical robot includes: a controller for controlling operation of the mobile surgical robot, a communications subsystem for communicating with the control station over the network, robotic surgical instruments controllable by the control station over the network, a detector subsystem for determining spatial information relating to a surgical environment of the surgical robot, and a motion stabilizer subsystem for facilitating operation of the robotic surgical instruments while the mobile surgical robot is in motion, wherein the controller is configured to operate a local control loop between at least one of the subsystems and the robotic surgical instruments. The control station includes: a control station controller for controlling operation of the control station, a control station communications subsystem for communicating with the mobile surgical robot over the network, and manipulation controllers for receiving manipulation inputs and for corresponding control of the robotic surgical instruments over the network.

In yet another example embodiment, the robotic surgical instruments of the mobile surgical robot are controlled using both master slave controls as well as intelligent automation.

In yet another example embodiment, the mobile surgical robot may be used to perform surgical procedures in a moving vehicle, including burr hole surgery, craniotomy surgery, treating haemorrhaging, and treating painful tumours.

### BRIEF DESCRIPTION OF THE DRAWINGS

Reference will now be made, by way of example, to the accompanying drawings which show example embodiments of the present application, and in which:

FIG. 1 shows a block diagram of a mobile robotic surgical system in accordance with an example embodiment;

FIG. 2 shows a perspective diagrammatic view of an example embodiment of the mobile robotic surgical system of FIG. 1;

FIG. 3 shows a perspective diagrammatic view of a control station to be used in the mobile robotic surgical system of FIG. 2;

FIG. 4 shows a perspective diagrammatic view of a mobile robotic platform to be used in the mobile robotic surgical system of FIG. 2 in operation;

FIG. 5 shows the mobile robotic platform of FIG. 4 in further operation;

FIG. 6 shows the mobile robotic platform of FIG. 5 in further operation;

FIG. 7 shows the mobile robotic platform of FIG. 6 in further operation;

FIG. 8 shows the mobile robotic platform of FIG. 7 in further operation;

FIG. 9 shows the mobile robotic platform of FIG. 8 in further operation;

FIG. 10 shows the mobile robotic platform of FIG. 9 in further operation;

FIG. 11, which illustrates an example library as stored in a storage of the mobile robotic platform.

Similar reference numerals may be used in different figures to denote similar components.

### DETAILED DESCRIPTION OF EXAMPLE EMBODIMENTS

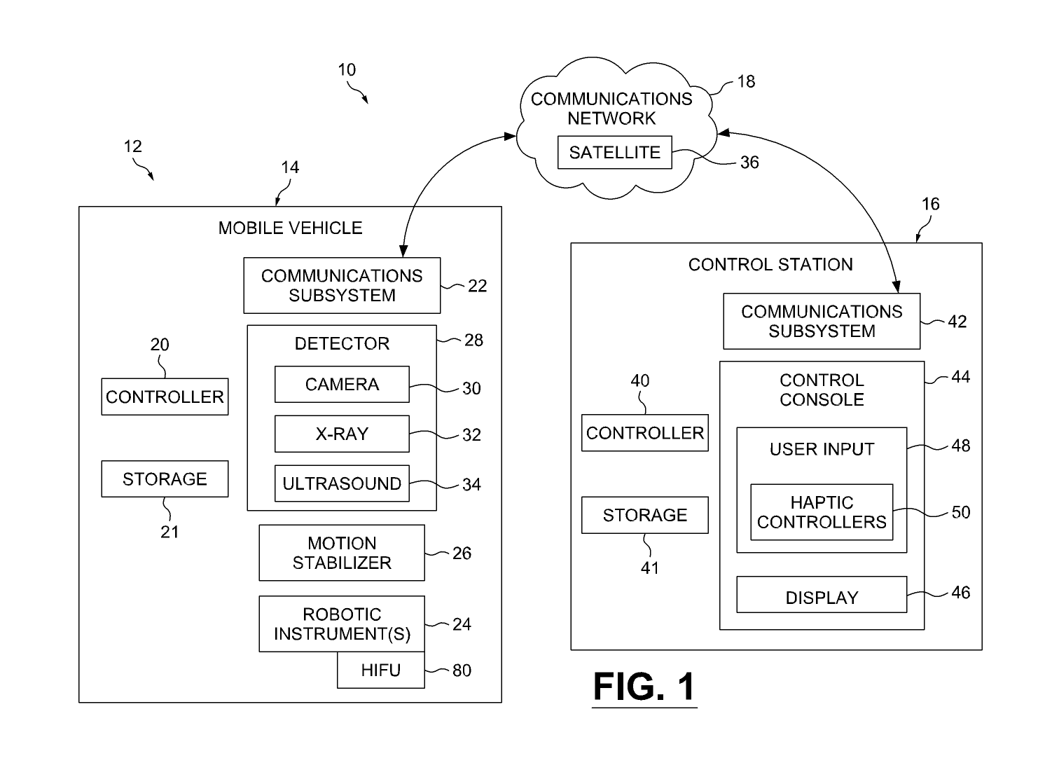

Reference is now made to FIG. 1, which shows a block diagram of a mobile robotic surgical system 10 in accordance with an example embodiment. The system 10 includes a mobile surgical robot 12 for use in a mobile environment such as mobile vehicle 14, or for use in other environments such as confined environments, remote locations, and hazardous or hostile areas. The mobile surgical robot 12 is in communication with a control station 16 located remotely to the mobile surgical robot 12. The mobile surgical robot 12 and the control station 16 are in communication over a communications network 18, which includes a satellite network 36. Generally, the mobile surgical robot 12 is operational while the mobile vehicle 14 is in motion or at the scene of the injury.

The mobile surgical robot 12 includes a controller 20 for controlling operation of the mobile surgical robot 12, a communications module or subsystem 22 for communicating with the control station 16 over the network 18, and robotic surgical instruments 24 haptically controllable by the control station 16 over the network 18. Reference to haptic includes force-feedback or touch-feedback control. The controller 20 can include one or more microprocessors that are coupled to a storage 21 that includes persistent and/or transient memory. The storage 21 stores information and software enabling the microprocessor(s) of controller 20 to control the subsystems and implement the functionality described herein. The mobile surgical robot 12 includes a motion stabilizer subsystem 26 for stabilizing or facilitating operation of the robotic surgical instruments 24 while the mobile vehicle 14 is in motion. The mobile surgical robot 12 also includes a detector subsystem 28 for determining spatial information relating to a surgical environment of the mobile surgical robot 12 (including a subject patient) and sending/relaying said information to the control station 16 over the network 18. As shown, in some example embodiments the detector 28 may include a camera 30 (for capturing video and/or audio information), an x-ray system 32, or an ultrasound system 34. The mobile vehicle 14 may include a conveyance or means of transport, for example including trucks, ambulances, trains, ships, aircraft and spacecraft. In some example embodiments, the controller 20 is configured to operate or provide a local control loop between at least one of the subsystems and the robotic surgical instruments 24.

The control station 16 includes a controller 40 for controlling operation of the control station 16 and a communications subsystem 42 for communicating with the mobile surgical robot 12 over the network 18. The controller 40 is coupled to a storage 41. A control console 44 provides an interface for interaction with a user, for example a surgeon. The control console 44 includes a display 46 (or multiple displays), and a user input 48. As shown, the user input 48 may include haptic controllers 50 for allowing the user to haptically control the robotic surgical instruments 24 of the mobile surgical robot 12.

Generally, the system 10 may be used to perform a procedure by breaking down a procedure into a series of interconnected sub-tasks. Some of the sub-tasks are performed automatically by the mobile surgical robot 12 to control the robotic instruments 24 and the subsystems to perform the particular sub-task. Some of the other sub-tasks are “semi-automated”, meaning having some control from the control station 16 as well as some local control from the controller 20. The particular allocation of sub-tasks for example assists when operating in the mobile vehicle 14, so that particular sub-tasks are performed as appropriate.

Each defined sub-task may for example be stored in a storage 21 accessible by the controller 20, the storage 21 including a library. The library includes a sequence of sub-tasks (both automated and “semi-atomated”). Specifically, some of the sub-tasks have instructions to automatically control the robotic instruments 24 and the subsystems to perform the sub-task. During automated control, the controller 20 may automatically perform the surgical functions by providing the local control loop with the subsystems. Some of the other sub-tasks may be “semi-automated”, meaning having some control from the control station 16 as well as some local automation (with the controller 20 providing local control loops as described herein). During semi-automated control, the control station 16 and the subsystems may be in a master-slave relationship. In example embodiments, such semi-automated control may be configured in an external control loop as between the subsystems and the robotic instruments 24, which are facilitated by the control station 16.

The sub-task may be selectively retrieved from the library and combined into a defined sequence or sequences to perform the surgical procedure. The flow from one sub-task to another is stored in the library. Each sub-task may use imagery and other parameters to verify sub-task completion. In some example embodiments, each of the sub-tasks in a particular entire procedure may be automatically performed by the mobile surgical robot 12.

In some example embodiments, a particular sub-task may be initially designated as “automated”, but may subsequently become or switch to semi-automated during the sub-task. For example, the operator at the control station 16 may override the automated mode based on viewing of the automated procedure on the display 46.

Similarly, a sub-task initially designated as “semi-automated” may subsequently become or switch to automated during the sub-task, and the controller 20 may override the remote control by the control station 16. Certain predetermined triggers detected by one of the subsystems may be used. For example, one of the subsystems in the surgical robot 12 may detect that the robotic instrument 24 is piercing the wrong tissue (based on a pre-stored expected tissue), which is detected by the controller 20, which may override to perform automatic control. Similarly, pre-stored images of the patient may be used to define “no-go” or partial “no-go” regions, and automatic control is triggered when the robotic instrument 24 enters such a region. In another example, the communications subsystem 22 may detect that communication to the control station 16 has been lost, or that network latency is beyond a predetermined threshold, thereby triggering an automatic control alert from the controller 20. In another example, the motion stabilizer subsystem 26 may detect that motion has exceeded a certain threshold, which is detected by the controller 20 to trigger automatic control.

In further example embodiments, the controller 20 may perform apportioning of control of the robotic surgical instruments between automatic control and semi-automatic control from the control station. For example, apportionment could initially be 50/50, but may change depending on various triggers detected by one or more of the subsystems.

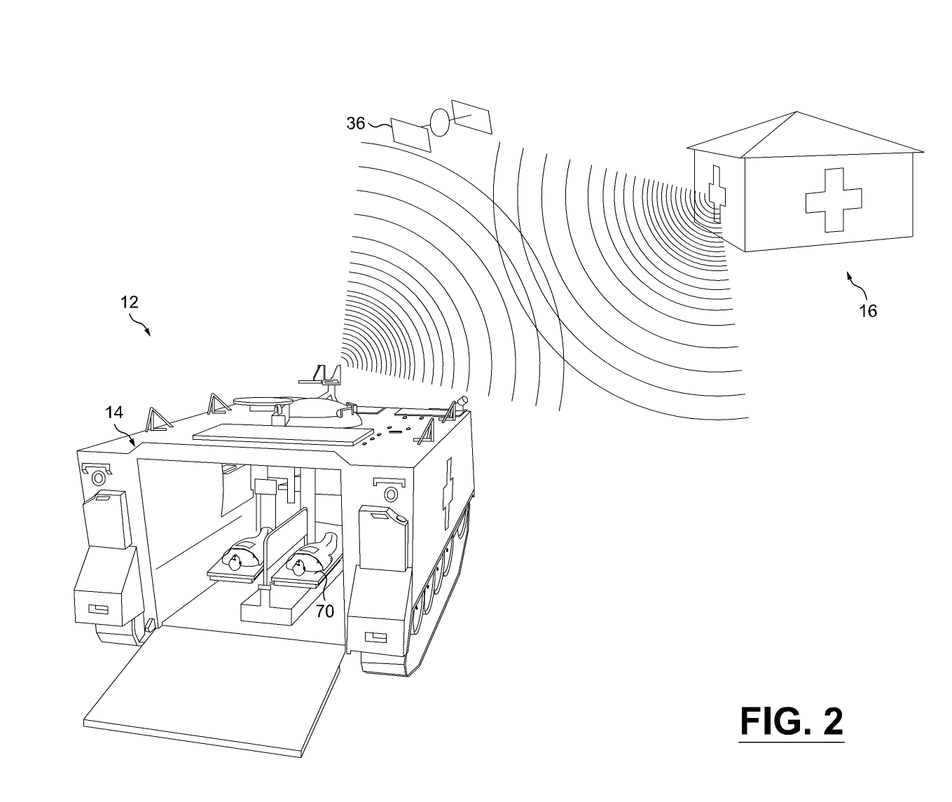

FIGS. 2 to 10 show an example embodiment of the system 10 of

FIG. 1. Referring briefly to FIG. 2, a subject patient may be provided on a platform 70 of the mobile vehicle 14. In the example shown, the mobile vehicle 14 is a military vehicle and the control station 16 is a medical treatment base, for performing surgical procedures on emergency or trauma patients.

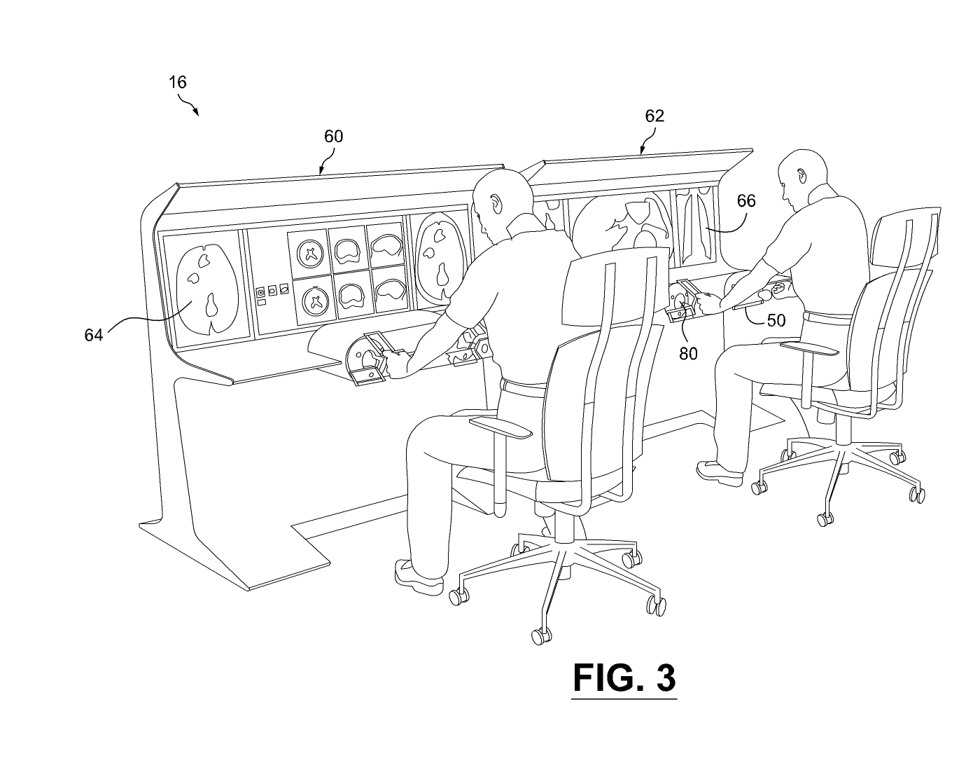

Reference is now made to FIG. 3, which shows the control station 16 in detail. The control console 44 may include first and second work stations 60, 62, as shown. Each workstation 60, 62 includes a display screen 64, 66 and left and right haptic controllers 50, which are manipulation controllers shown as stylus gimbals to allow the operator (e.g. surgeon) to manipulate and control each workstation 60, 62. The haptic controllers 50 provide touch feedback to the operator(s) based on forces sensed force sensors located within the robotic surgical instruments 24 within the mobile vehicle 14 (FIG. 1). Both workstations 60, 62 may for example control separate robotic instruments 24 (FIG. 1), or may work together to control the same robotic instruments 24, for example in a master/slave configuration which may for example be used for training. Such a training system is described in detail by the Applicant in PCT/CA2007/000676, published Nov. 1, 2007, the contents of which are hereby incorporated by reference. The particular configuration and operation of the haptic controllers 50 is dependent on the particular application of the system 10.

The workstations 60, 62 may also be configured to define the work envelope of the corresponding surgical robotic instruments 24, work within and keep out zones for single arm and multi arm surgical robotics. This data may be used in developing collision avoidance algorithms that will be incorporated into the software for robotic control. Example implementations are also described in PCT Application No. PCT/CA2007/000676.

Referring again to FIG. 1, the robotic surgical instruments 24 may include any number or combination of controllable mechanisms. The robotic surgical instruments 24 include end effectors such as grippers, cutters, manipulators, forceps, bi-polar cutters, ultrasonic grippers & probes, cauterizing tools, suturing devices, etc. The robotic surgical instruments 24 generally include small lightweight actuators and components. In some example embodiments, the robotic surgical instruments 24 include pneumatic and/or hydraulic actuators. Such actuators may further assist in providing motion stability, as further described below. In some example embodiments, various lightweight radiolucent materials for robotic arms as well as the range joint torques, forces, frequency response, ROM, weight and size of different actuators to achieve the maximum function in the mobile surgical robot 12.

Referring now to the motion stabilizer subsystem 26, in some example embodiments the motion stabilizer subsystem 26 provides motion isolation from motion of the mobile vehicle 14 using either magnetic or Lorentz levitation technology, as would be understood by those skilled in the art. In another example embodiment, the motion stabilizer subsystem 26 includes a motion sensor which detects the induced motion of the mobile surgical robot 12 associated with vehicle motion and provides a compensating or restraining force to the robotic surgical instruments 24 in response, to reduce relative motion between the patient and the robotic surgical instrumentation. In such embodiments, active control may be used to implement such a system. The motion sensors may include one or more accelerometers to detect vehicle acceleration, deceleration, dynamics and characteristics of motion aboard the mobile vehicle 13. The particular motion stabilizer subsystem 26 used depends on the particular application of the system 10.

In some example embodiments, the motion stabilizer subsystem 26 includes a control loop force feedback (e.g., implemented by the controller 20 within the mobile surgical robot 12) to prevent the robotic surgical instruments 24 from imparting forces beyond a predetermined threshold, for example for the force not to exceed a threshold on soft tissue and bone while the mobile vehicle 14 is in motion. The motion stabilizer subsystem 26 may includes force sensors, which in some example embodiments be located on the robotic surgical instruments 24 themselves. It can be appreciated that the range of force imparted may depend on the particular subject tissue being operated on. In example embodiments, the controller 20 can compare an expected particular subject tissue (the parameters of which may be stored within the storage 21) with the actual detected tissue.

Referring still to FIG. 1, the detector subsystem 28 will now be described in greater detail. The incorporation of intra-operative image guidance into surgical robotics provides an additional capability to refine the precision of a surgical procedure. Pre-operative diagnostic imagery may be utilized to plan surgical procedures with the assumption that these diagnostic images will represent tissue morphology at the time of surgery. Along with this pre-operative planning, intra-operative imagery may also be used to modify or refine a present surgical procedure or administer minimally invasive treatment such as HIFU ultrasound therapy used to control bleeding.

One aspect of such image-guided surgery in accordance with example embodiments is registering multiple images to each other and to the patient, tracking instruments intra-operatively and subsequently translating this imagery for real time use in the robot space. The incorporation of medical imagery into surgical planning for the present system 10 facilitates the identification of a defined work envelope for single or multiple robotic arms. Intra-operative tracking of the position of the robotic surgical instruments 24 within the defined work envelope can be utilized to develop local control loop systems between the detector 28 and the robotic surgical instruments 24 to define keep-out and work within zones for surgical tasks. This data is incorporated into known algorithms developed for collision avoidance of the multiple robotic arms and optimization of the position of instrumentation for completion of the surgical task.

Different technologies that incorporate a mechanical linkage, such as IR (Infrared) markers or RF (Radiofrequency) devices may be used for image registration of specific anatomical landmarks for both the intra-operative tracking of a surgical robot in relation to the patient as well as tracking the surgical instrumentation. Image-based registration is less sensitive to calibration and tracking errors as it provides a direct transformation between the image space and the instrument space. The information from anatomical landmarks can be registered with the diagnostic imagery used to plan the surgical procedure and subsequently translated into the robotic space for completion of an image guided surgical procedure. This translation is performed using a registration procedure between the robot and the imaging device. The incorporation of real-time intra-operative tracking of anatomical landmarks provides a mechanism of incorporating compensatory motion of the robotic arm to accommodate patient movement thereby enhancing the precision of the robotic task.

In another example embodiment, the detector subsystem 28 includes the incorporation of image guidance into the robotic surgery, including predetermined marker shapes and positions that provide optimal accuracy for IR monitoring and tracking of anatomical landmarks, instrument position and the position of the robotic arms under the constraints imposed by the imaging device and the limited volume available in the surgical work envelope.

In another example embodiment, the detector subsystem 28 includes a number of ceiling mounted cameras 30 and two small X-ray machines 32 inside the mobile vehicle, which can take two 2-D images, at for example 30 degree angles which allow computer renderings into 3D image for surgeon use.

Imagery can also be incorporated as one of many parameters used to provide local control loop feedback in performing autonomous robotic tasks. In some example embodiments, the control station 16 and mobile surgical robot 12 operate in a master slave relationship. Such embodiments may incorporate semi-autonomous surgical robotics wherein the mobile surgical robot 12 may autonomously perform some specified surgical tasks that are part of a sequence of a larger task comprising the surgical procedure, for example using a locally controlled loop implemented by the controller 20. This may for example enables the surgeon to selectively perform techniques best undertaken with a master slave relationship while using automated robotics to perform specific tasks that require the enhanced precision of a surgical robot. For example, such tasks may include the precision placement of brachytherapy for cancer treatment or the precision drilling and intra-operative positioning of hardware in orthopaedic surgery.

Referring still to FIG. 1, the communications network 18 may further include a direct wireless connection, a wide area network such as the Internet, a wireless wide area packet data network, a voice and data network, a public switched telephone network, a wireless local area network (WLAN), or other networks or combinations of the forgoing. It has been thought that the incorporation of haptic feedback into surgical robotics has proved challenging due to the need for short latencies, often less than 50 milliseconds, required for high fidelity haptic feedback.

In some example embodiments, the communications subsystems 22, 42 communicate over the satellite network 36, which may for example include incorporation of a C band satellite telecommunications infrastructure to support the communication therebetween. Generally, in some embodiments the system 10 may readily perform with network latencies of less than 300 milliseconds. In some example embodiments, the system may use longer latencies up to 700 milliseconds with a tradeoff of both an increase in task completion time and error rate. Longer latencies may also be implemented.

Use of the satellite network 36 may be beneficial as many remote environments lack sophisticated terrestrial telecommunications capability. Satellite technology can also be used in the event of natural disasters.

In some example embodiments, redundant telecommunication functionality is used to eliminate single point failures and create redundancy to provide seamlessly integrated into the telecommunications interface. For example, a combination of satellite and public land mobile networks (PLMN) may be used.

Referring now to FIGS. 2 to 7, an example embodiment will be described. The concept of dividing a surgical procedure into a sequence of sub-tasks with the use of imaging to assess sub-task completion can be used to develop some autonomy in surgical robotics. These sub-tasks constitute sequential steps in a surgical procedure that can be linked together. Each step in the sequence of sub-tasks incorporates control loop feedback to verify successful completion before proceeding to the next sub-task. The successful completion of the series of sub-tasks constitutes completion of the surgical procedure. Depending upon the type of surgical procedure, intra-operative CT or x-ray 32 imagery may for example be used to assess completion of the sub-tasks. The intra-operative imagery for sub-task verification may be augmented by control-loop feedback from the other subsystems in the mobile surgical robot 12. The semi-autonomous and autonomous capability in surgical robotics is incorporated into the mobile surgical robot 12. Such features may for example address problems with variable or prolonged signal latencies or when data is corrupted by signal loss, disruption or electrical noise.

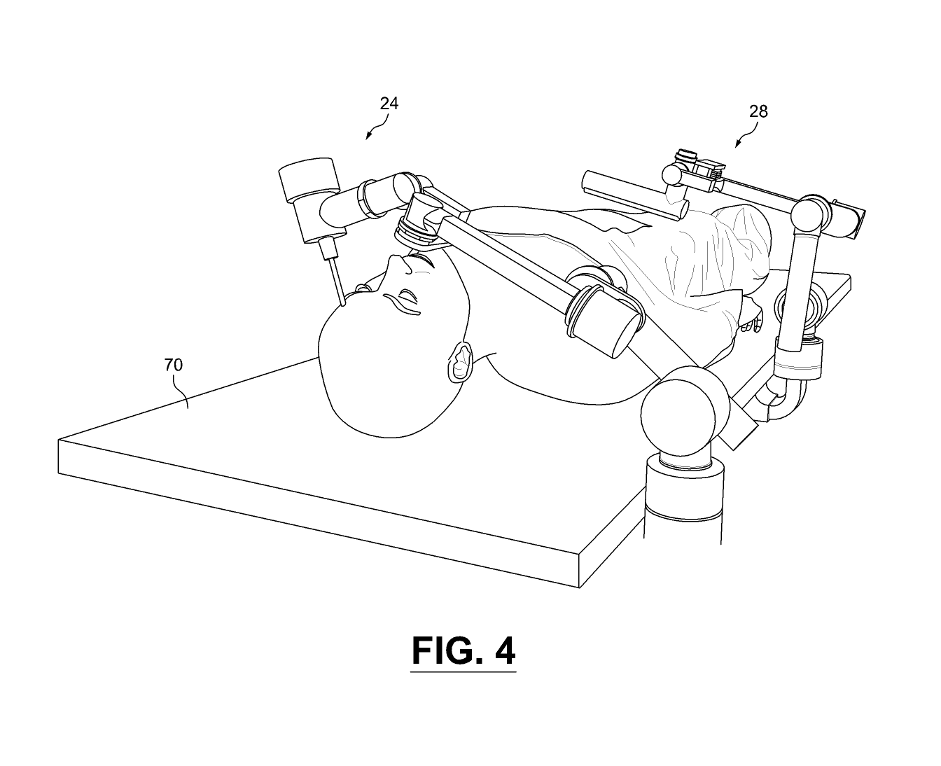

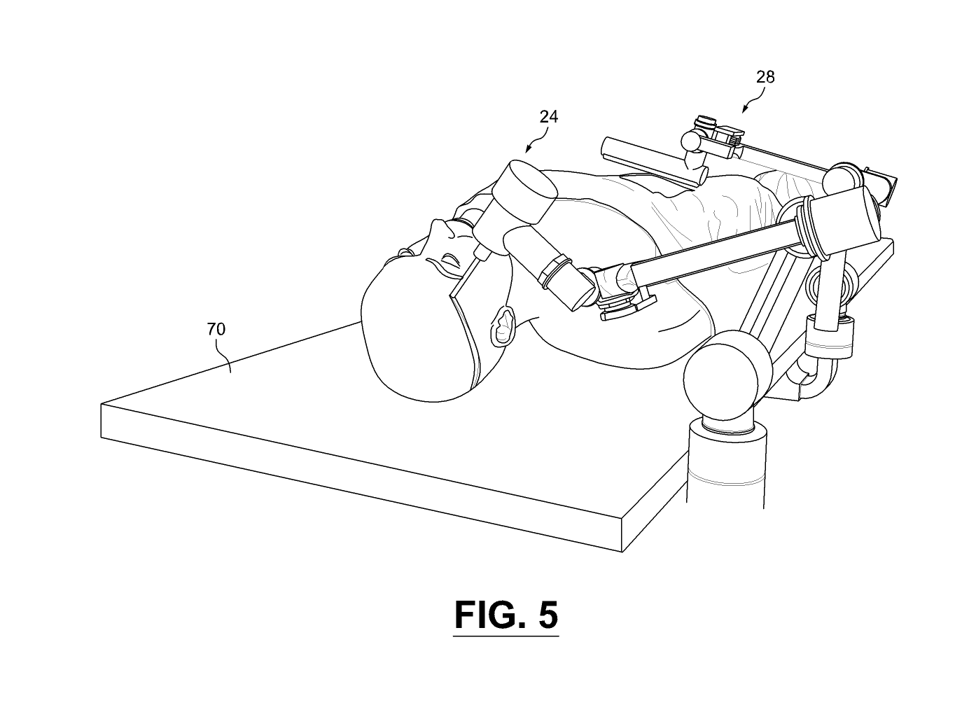

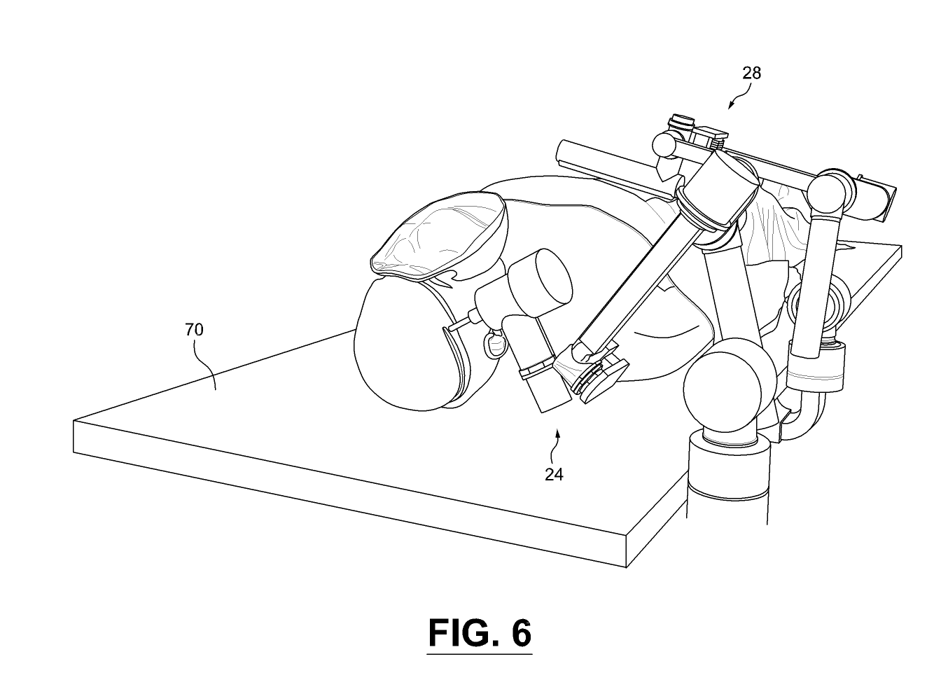

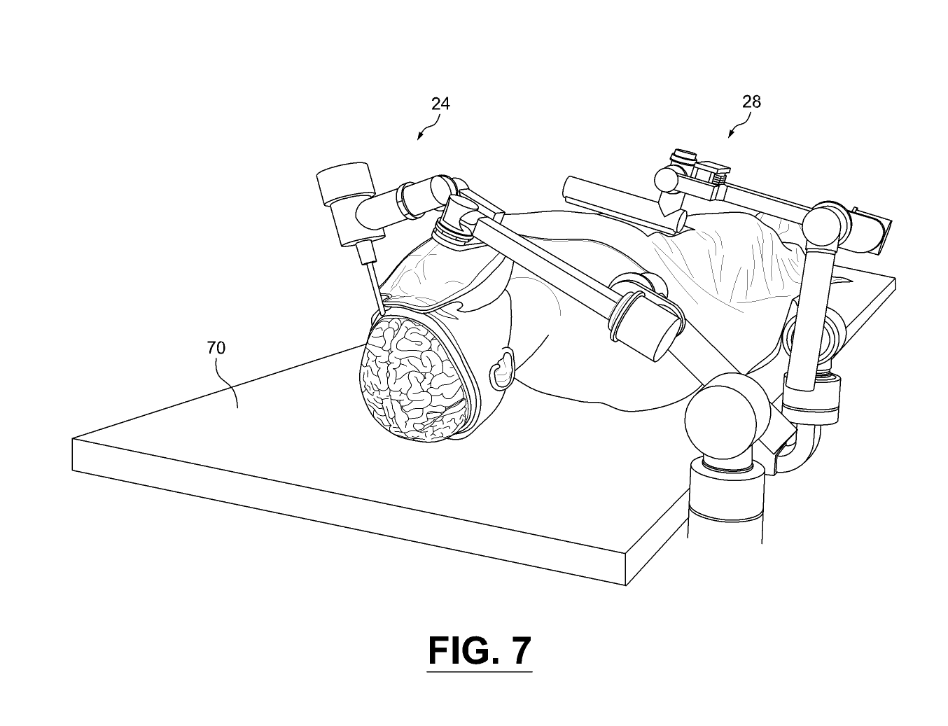

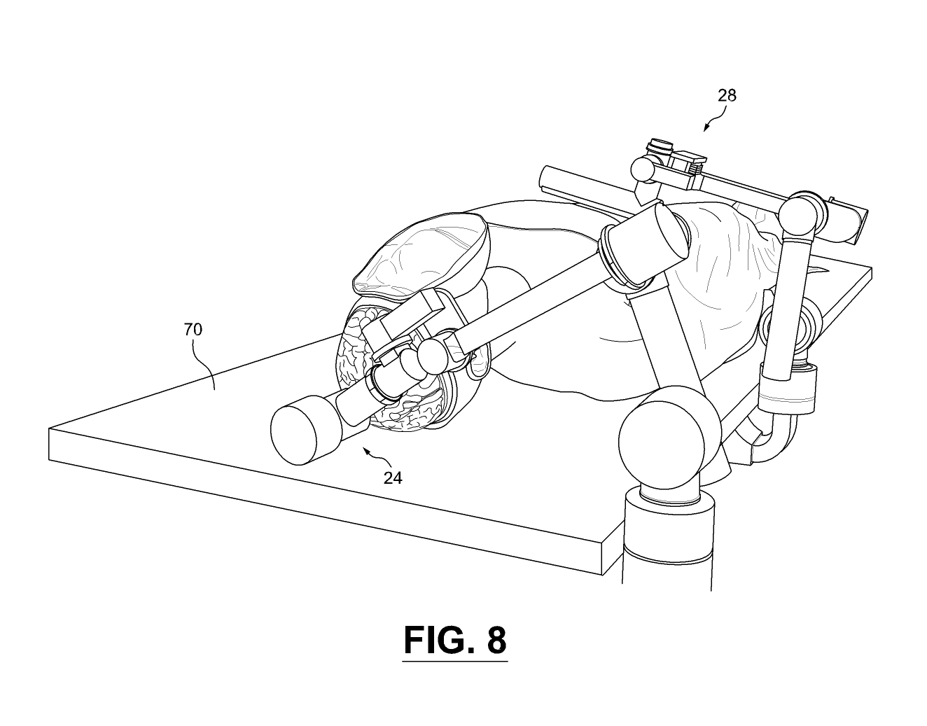

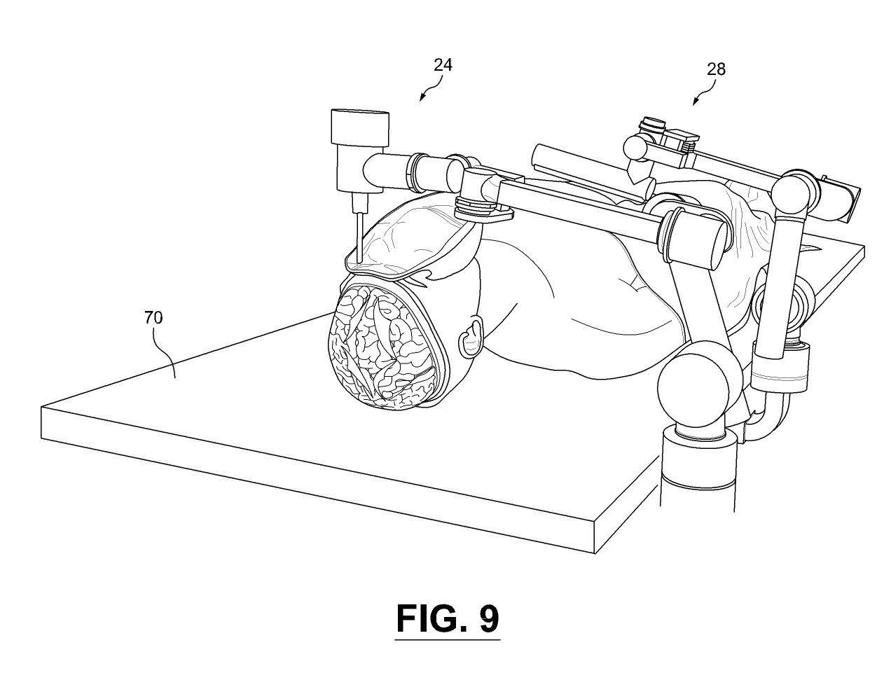

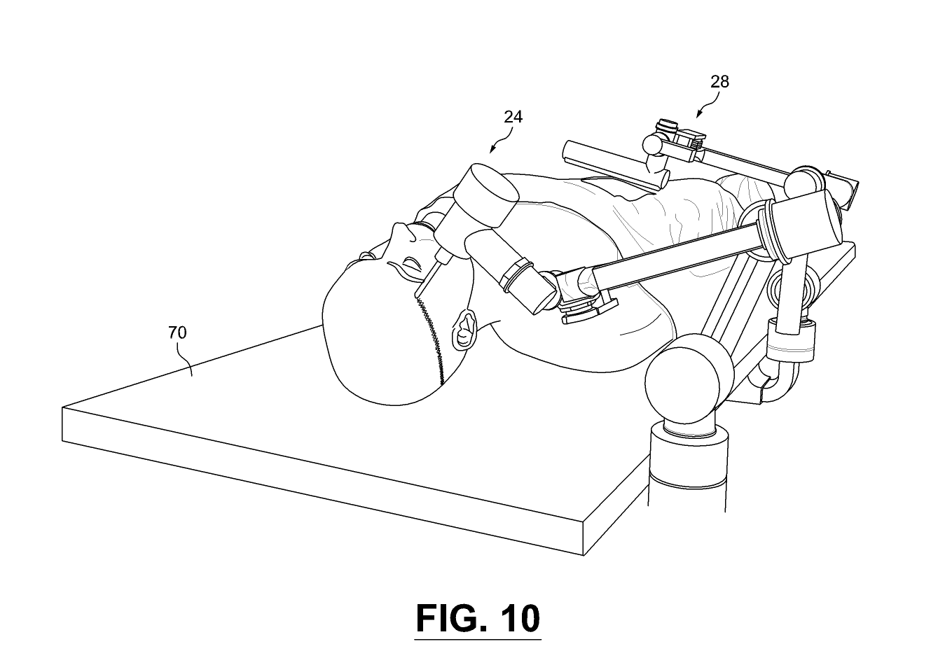

In the example shown, a craniotomy (or in the simpler case a surgical burr hole used clinically to drain an acute epidural hematoma), may be implemented by the system 10. The particular procedure may be broken down into a series of interconnected sub-tasks defined by and integrated with the detector subsystem 28 in a localized control loop. Although the detector subsystem 28 is shown in the drawings as being generally pointed at the abdomen for ease of illustration, it can be appreciated that the detector subsystem 28 may point at any or all areas of the patient or surgical environment. In the example shown, FIG. 4 shows the robotic surgical instruments 24 in position at the skull. FIG. 5 shows the robotic surgical instruments 24 drilling or cutting the skin of the patient. FIG. 6 shows the robotic surgical instruments 24 drilling or cutting the skull of the patient. FIG. 7 shows the robotic surgical instruments 24 removing or lifting the skull from the patient. FIGS. 8 and 9 show the specific craniometry procedure being performed by the robotic surgical instruments 24. FIG. 10 shows the robotic surgical instruments 24 suturing the skin of the patient.

In the simpler example of a burr hole, some standard anatomical landmarks may be used to locate the position of a burr hole on the cranium for placement of IR markers for registration into the robot space. In example embodiments, the procedure may initially be semi-automated, wherein the operator positions the drill over the appropriate position, for example by moving the drill to the skull in position to drill, without piercing the bone or tissue. The markers are used by the operator to verify the position of the robotic end effector that holds the surgical drill at commencement of the sub-task. The next sub-task is to drill through the skull. This may be either semi-automated or full automated by the surgical robot 12. The local loop control is thus used to facilitate the sub-task of drilling. In some example embodiments, real-time monitoring of running torque and local temperature (or other sensors which may for example be located within the robotic instrument(s) 24) are use to provide additional information feedback for local loop control of drilling through the skull. Upon successful completion of drilling, the next sub-task is for removal of the bone plug. This may for example be semi-automated as well.

An appropriate array of end effectors for soft tissue manipulation and surgical drilling may be autonomously selected and utilized by the mobile surgical robot 12 to complete the drilling procedure. A suitable component of the end effector may be used for removing of the bone plug.

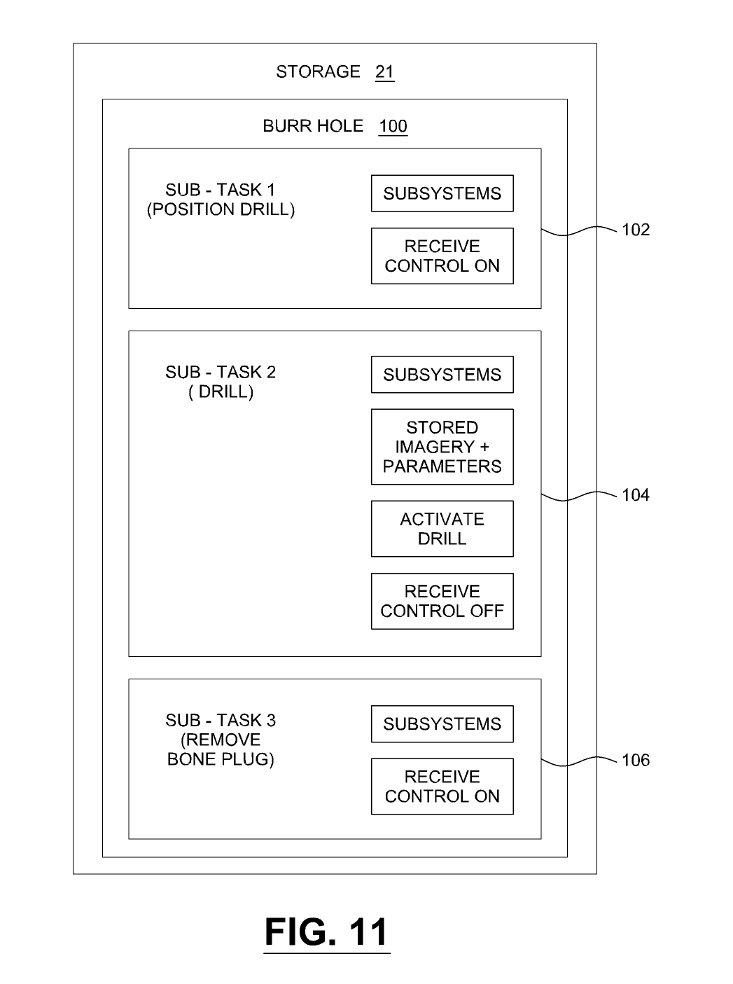

Reference is now made to FIG. 11, which illustrates an example library as stored in the storage 21. The example library shown includes modules or instructions for an example burr hole procedure 100. As shown, the burr hole procedure may include three sub-tasks being sub-task1 (102), sub-task2 (104), and sub-task3 (106). In the example shown, sub-task1 (102) is for positioning of the drill, sub-task2 (104) is for drilling through the skull, and sub-task3 (106) is for removing of the bone plug. As shown, sub-task1 (102) includes instructions for activating the subsystems and turning on receive control from the control station 16. Sub-task2 (104) includes instructions for activating the subsystems, accessing stored imagery and parameters (for providing a reference for the drilling), controlling the robotic instruments 24 (e.g., a drill). and turning off receive control from the control station 16. Sub-task3 (106) includes instructions for activating the subsystems and turning on receive control from the control station 16. In example embodiments, the toggling of receive control from the control station 16 to ON or OFF may be activated based on instructions or alerts from one of the subsystems of the mobile surgical robot 12 or from the control station 16.

Similarly, in example embodiments, storage 21 may contain a library of sub-tasks (not shown) for a craniotomy, which may include automated and semi-automated instructions in a similar fashion.

Referring to FIG. 1, in another example embodiment, the robotic surgical instrument 24 may be configured to include a therapeutic tool utilizing the administration of high intensity focused ultrasound (HIFU) 80 to control haemorrhage and treat solid tumours. Automated algorithms may be used to assist in the diagnosis of solid tumours and may be of value in aiding the diagnosis of intra-abdominal or pelvic haemorrhage following blunt abdominal trauma. The non-invasive nature of ultrasound imaging supports the development of an automated command sequence for autonomous robotic abdominal ultrasonography to detect splenic injury following blunt abdominal trauma. Identification of the site of splenic injury through ultrasonography can then be incorporated into a programmed sequence of subtasks designed to administer HIFU 80 for haemorrhage control. In some example embodiments, such a system includes storing diagnostic images with key anatomical features, registering this in the robot space and then using the data for precision administration of HIFU 80. Different high frequency ultrasound settings may be used to accomplish homeostasis following traumatic injury to soft tissues and spleen, depending on the particular application. Both the HIFU 80 and the ultrasound 34 (for detecting the surgical environment) may be implemented within the same robotic surgical instrument 24.

In some example embodiments, the system 10 may further include quick disconnect technologies for power and data connectivity with conventional mobile vehicles 14. The system may be modular or permit retro-fitting of existing mobile vehicles 14, for example an ambulance or military vehicle.

In some example embodiments, the mobile surgical robot 12 is itself a moving vehicle, and may for example include its own wheels and motor control for moving.

In another example embodiment, the system 10 may be used for integrated digital radiography to diagnose extremity, pelvic, spinal fractures.

In another example embodiment, the system 10 may be used for placement of interosseous infusion device.

In another example embodiment, the system 10 may be used for placement of temporary external fixation for unstable extremity and pelvic fractures.

In another example embodiment, the system 10 may be used for placement of halo stabilization device for unstable cervical spine injuries.

In another example embodiment, the system 10 may be used for diagnosis and treatment of blunt splenic injury.

In another example embodiment, the system 10 may be used for needle decompression of tension pneumothorax.

In another example embodiment, the system 10 may include diagnostic or monitoring systems integrated into telementoring software package using USB connectivity to pulse oximeters, electronic stethoscopes, EKG, IV constant infusion pumps, to enable diagnosis and resuscitation of physiologically unstable medical or surgical patients.

In another example embodiment, the system 10 may include pneumatic splinting systems for stabilizing the patient, pelvis, extremities as needed during transport. These pneumatic splints utilize local control loop feedback from pressure sensors to prevent over-inflation with air expansion within the splint that occurs during aeromedical evacuation.

In another example embodiment, there is provided a mobile surgical robot, including: a controller for controlling operation of the mobile surgical robot; a communications subsystem for communicating over a network with a control station located remotely to the mobile surgical robot; robotic surgical instruments controllable by the control station over the network; a detector subsystem for determining spatial information relating to a surgical environment of the mobile surgical robot; and a motion stabilizer subsystem for facilitating operation of the robotic surgical instruments while the mobile surgical robot is in motion, wherein the controller is configured to operate a local control loop between at least one of the subsystems and the robotic surgical instruments.

In another example embodiment, there is provided a method for controlling a mobile surgical robot. The method includes: controlling operation of the mobile surgical robot using a controller; communicating with a control station located remotely to the mobile surgical robot over a network using a communications subsystem; receiving commands from the control station over the network for controlling robotic surgical instruments of the mobile surgical robot; determining spatial information relating to a surgical environment of the mobile surgical robot using a detector subsystem; facilitating operation of the robotic surgical instruments while the mobile surgical robot is in motion using a motion stabilizer subsystem; and operating a local control loop between at least one of the subsystems and the robotic surgical instruments using the controller.

In another example embodiment, there is provided a mobile robotic surgical system, comprising a mobile surgical robot and a control station located remotely to the mobile surgical robot in communication with the mobile surgical robot over a network. The mobile surgical robot includes: a controller for controlling operation of the mobile surgical robot, a communications subsystem for communicating with the control station over the network, robotic surgical instruments controllable by the control station over the network, a detector subsystem for determining spatial information relating to a surgical environment of the surgical robot, and a motion stabilizer subsystem for facilitating operation of the robotic surgical instruments while the mobile surgical robot is in motion, wherein the controller is configured to operate a local control loop between at least one of the subsystems and the robotic surgical instruments. The control station includes: a control station controller for controlling operation of the control station, a control station communications subsystem for communicating with the mobile surgical robot over the network, and manipulation controllers for receiving manipulation inputs and for corresponding control of the robotic surgical instruments over the network.

In another example embodiment, the manipulation controllers in the control station include haptic controllers for haptically controlling the robotic surgical instruments.

Variations may be made to some example embodiments, which may include combinations and sub-combinations of any of the above. The various embodiments presented above are merely examples and are in no way meant to limit the scope of this disclosure. Variations of the innovations described herein will be apparent to persons of ordinary skill in the art, such variations being within the intended scope of the present disclosure. In particular, features from one or more of the above-described embodiments may be selected to create alternative embodiments comprised of a sub-combination of features which may not be explicitly described above. In addition, features from one or more of the above-described embodiments may be selected and combined to create alternative embodiments comprised of a combination of features which may not be explicitly described above. Features suitable for such combinations and sub-combinations would be readily apparent to persons skilled in the art upon review of the present disclosure as a whole. The subject matter described herein intends to cover and embrace all suitable changes in technology.

What is claimed is:

1. A mobile surgical robot, comprising:
a controller for controlling operation of the mobile surgical robot;
a communications subsystem for communicating over a network with a control station located remotely to the mobile surgical robot;
robotic surgical instruments controllable by the control station over the network;
a detector subsystem for determining spatial information relating to a surgical environment of the mobile surgical robot; and
a motion stabilizer subsystem for facilitating operation of the robotic surgical instruments while the mobile surgical robot is in motion,
wherein the controller is configured to operate a local control loop between at least one of the subsystems and the robotic surgical instruments.

2. The mobile surgical robot as claimed in claim 1, wherein said at least one of the subsystems and the robotic surgical instruments are further operable by the control station in an external control loop.

3. The mobile surgical robot as claimed in claim 1, further comprising a memory, wherein the controller is configured to access the memory to control the robotic surgical instruments and said at least one subsystem.

4. The mobile surgical robot as claimed in claim 3, wherein the memory includes a library of sub-tasks to be performed.

5. The mobile surgical robot as claimed in claim 4, wherein one or more of the sub-tasks include instructions for automatically controlling the robotic surgical instruments and said at least one subsystem using the local control loop.

6. The mobile surgical robot as claimed in claim 4, wherein one or more of the sub-tasks include robotic surgical instruments and said at least one subsystems being controlled by the control station in an external control loop.

7. The mobile surgical robot as claimed in claim 1, wherein the controller is further configured for switching control of the robotic surgical instruments between automatic control using the local control loop and control from the control station.

8. The mobile surgical robot as claimed in claim 7, wherein said switching is performed by the controller based on detection of a predetermined event detected from one of the subsystems.

9. The mobile surgical robot as claimed in claim 1, wherein the controller is further configured for apportioning control of the robotic surgical instruments between automatic control using the local control loop and control from the control station.

10. The mobile surgical robot as claimed in claim 1, wherein the motion stabilizing subsystem includes a motion sensor for detecting motion of the mobile surgical robot.

11. The mobile surgical robot as claimed in claim 1, wherein the network includes a network latency.

12. The mobile surgical robot as claimed in claim 1, wherein the network includes a satellite system, and the communications subsystem communicates over the network using a satellite compatible communications protocol.

13. The mobile surgical robot as claimed in claim 1, wherein the controller is further configured to send spatial information detected by the detector subsystem to the control station over the network.

14. A method for controlling a mobile surgical robot, the method comprising:
controlling operation of the mobile surgical robot using a controller;
communicating with a control station located remotely to the mobile surgical robot over a network using a communications subsystem;
receiving commands from the control station over the network for controlling robotic surgical instruments of the mobile surgical robot;
determining spatial information relating to a surgical environment of the mobile surgical robot using a detector subsystem;
facilitating operation of the robotic surgical instruments while the mobile surgical robot is in motion using a motion stabilizer subsystem; and
operating a local control loop between at least one of the subsystems and the robotic surgical instruments using the controller.

15. The method as claimed in claim 14, further comprising operating said at least one of the subsystems and the robotic surgical instruments using the control station in an external control loop.

16. The method as claimed in claim 14, further comprising accessing a memory using the controller to control the robotic surgical instruments and said at least one subsystem.

17. The method as claimed in claim 16, wherein the memory includes a library of sub-tasks to be performed.

18. The method as claimed in claim 14, wherein one or more of the sub-tasks include instructions for automatically controlling the robotic surgical instruments and said at least one subsystem using the local control loop.

19. The method as claimed in claim 14, wherein one or more of the sub-tasks include robotic surgical instruments and said at least one subsystem being controlled by the control station in an external control loop.

20. The method as claimed in claim 14, further comprising switching control of the robotic surgical instruments between automatic control using the local control loop and control from the control station.

21. The method as claimed in claim 20, wherein said switching is performed by the controller based on detection of a predetermined event detected from one of the subsystems.

22. The method as claimed in claim 14, further comprising apportioning control of the robotic surgical instruments between automatic control using the local control loop and control from the control station.

23. A mobile robotic surgical system, comprising:
a mobile surgical robot; and
a control station located remotely to the mobile surgical robot in communication with the mobile surgical robot over a network,
wherein the mobile surgical robot includes:
a controller for controlling operation of the mobile surgical robot,
a communications subsystem for communicating with the control station over the network,
robotic surgical instruments controllable by the control station over the network,
a detector subsystem for determining spatial information relating to a surgical environment of the surgical robot, and
a motion stabilizer subsystem for facilitating operation of the robotic surgical instruments while the mobile surgical robot is in motion, wherein the controller is configured to operate a local control loop between at least one of the subsystems and the robotic surgical instruments,
wherein the control station includes:
a control station controller for controlling operation of the control station,
a control station communications subsystem for communicating with the mobile surgical robot over the network, and
manipulation controllers for receiving manipulation inputs and for corresponding control of the robotic surgical instruments over the network.

24. The mobile robotic surgical system of claim 23, wherein the manipulation controllers in the control station include haptic controllers for haptically controlling the robotic surgical instruments.
